(19) United States
(12) Plant Patent Application Publication
(10) Pub. No.: US 2017/0215318 P1
(43) Pub. Date: Jul. 27, 2017

(54) Argyranthemum plant named 'SUN 480'

(50) ;

(71) Applicant: Josef Heuger, Glandorf (DE)

(72) Inventor: Josef Heuger, Glandorf (DE)

(21) Appl. No.: 14/998,612

(22) Filed: Jan. 23, 2016

(51) Int. Cl. A01H 5/02

(52) U.S. Cl. PLT/406

(57) ABSTRACT

A new and distinct cultivar of plant named ‘SUN 480’, characterized by its uniformly mounded plant habit; freely branching habit; freely flowering habit; single-type inflorescences with dark red-colored ray florets; and good garden performance.

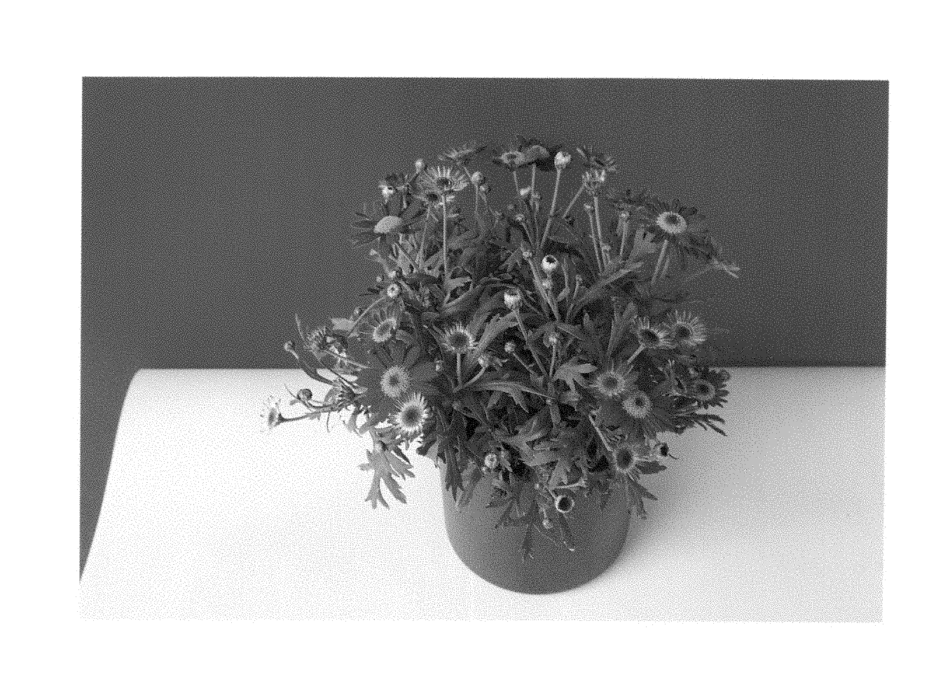

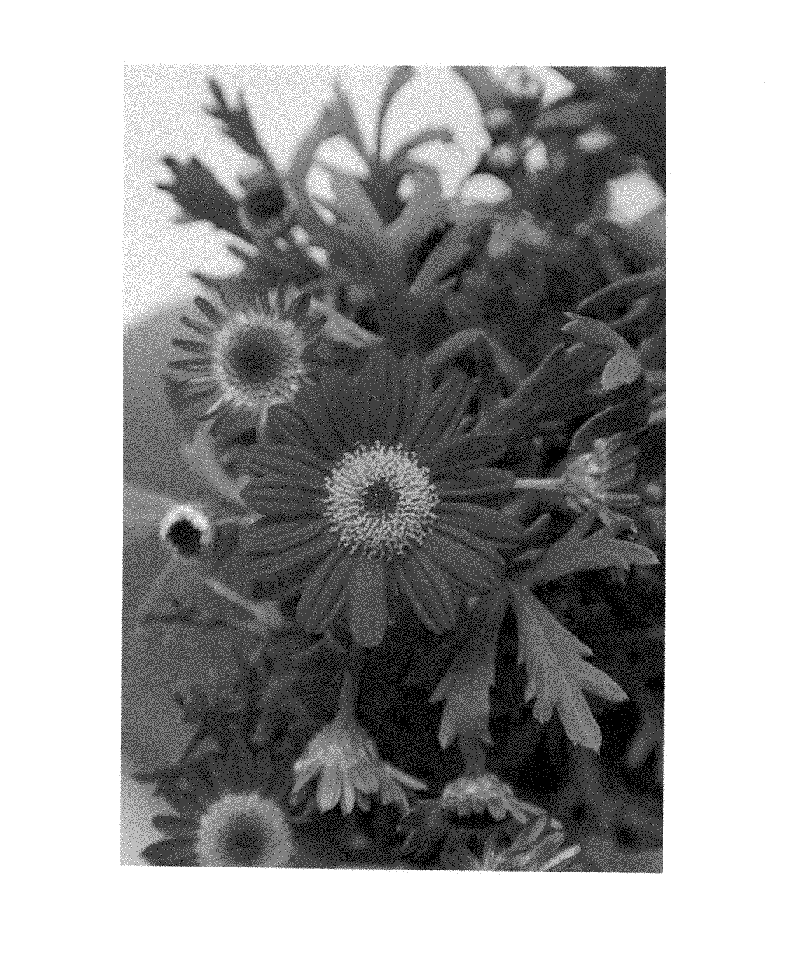

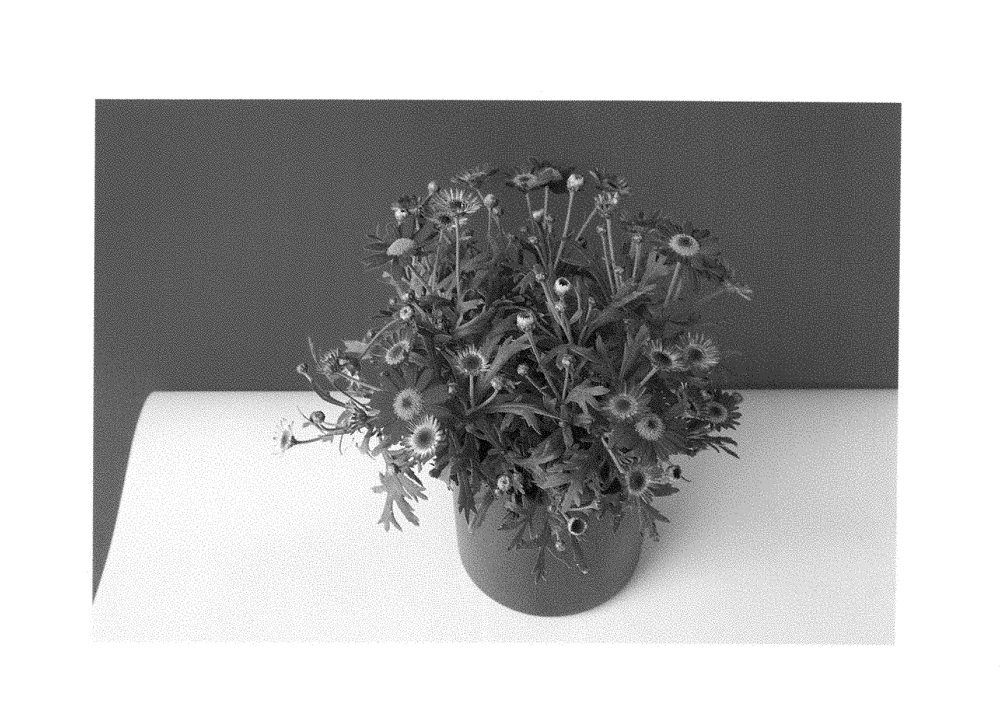

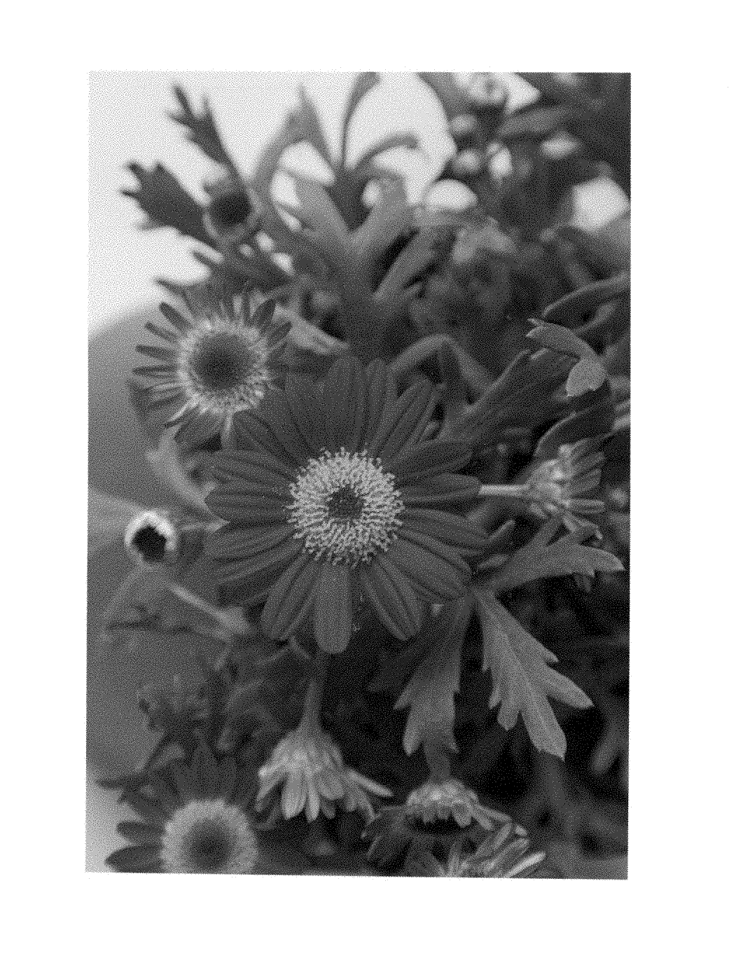

Botanical designation: Argyranthemum frutescens.

Cultivar denomination: ‘SUN 480’.

### BACKGROUND OF THE INVENTION

The present invention relates to a new and distinct cultivar of Argyranthemum plant, botanically known as Argyranthemum frutescens and hereinafter referred to by the cultivar name ‘SUN 480’.

The objective of the breeding program is to create new Argyranthemum plants with attractive ray and disc floret colors and good garden performance and pest resistance.

The new Argyranthemum plant originated from a cross-pollination made by the Inventor in July, 2012 in Glandorf, Germany of two unnamed seedling selections of Argyranthemum frutescens, not patented. The new Argyranthemum plant was discovered and selected by the Inventor as a single flowering plant from within the progeny of the stated cross-pollination in a controlled greenhouse environment in Glandorf, Germany in July, 2013.

Asexual reproduction of the new Argyranthemum plant by vegetative tip cuttings was first conducted in Glandorf, Germany in February, 2014. Asexual reproduction by cuttings has shown that the unique features of this new Argyranthemum plant are stable and reproduced true to type in successive generations.

### SUMMARY OF THE INVENTION

Plants of the new Argyranthemum have not been observed under all possible combinations of environmental conditions and cultural practices. The phenotype may vary somewhat with variations in environmental conditions such as temperature, daylength and light intensity, without, however, any variance in genotype.

The following traits have been repeatedly observed and are determined to be the unique characteristics of the new Argyranthemum plant. These characteristics in combination distinguish ‘SUN 480’ as a new and distinct Argyranthemum plant:
1. Uniformly mounded plant habit.
2. Freely branching habit.
3. Freely flowering habit.
4. Single-type inflorescences with dark red-colored ray florets.
5. Good garden performance.

Plants of the new Argyranthemum differ from plants of the parent selections primarily in uniformity as plants of the new Argyranthemum are more uniformly mounding than plants of the parent selections.

Plants of the new Argyranthemum can be compared to plants of Argyranthemum frutescens ‘SUN 20’, not patented. In side-by-side comparisons conducted in Glandorf, Germany, plants of the new Argyranthemum differed primarily from plants of ‘SUN 20’ in ray floret color as plants of ‘SUN 20’ have pink-colored ray florets.

### BRIEF DESCRIPTION OF THE PHOTOGRAPHS

The accompanying photographs illustrate the overall appearance of the new Argyranthemum showing the colors as true as it is reasonably possible to obtain in colored reproductions of this type. Colors in the photographs may differ slightly from the color values cited in the detailed botanical description which accurately describe the colors of the new Argyranthemum plant. The photograph on the first sheet comprises a side perspective view of a typical flowering plant of ‘SUN 480’ grown in a container. The photograph on the second sheet is a close-up view of a typical flowering plant of ‘SUN 480’.

### DETAILED BOTANICAL DESCRIPTION

The aforementioned photographs and following observations and measurements describe plants grown during the spring and early summer in 9-cm containers in a glass-covered greenhouse in Glandorf, Germany and under cultural practices typical of commercial potted Argyranthemum production. During the production of the plants, day temperatures ranged from 14° C. to 28° C., night temperatures ranged from 10° C. to 18° C. and light levels ranged from 25 klux to 90 klux. Plants were pinched one time five weeks after planting and were 2.5 months old when the photographs and description were taken. In the following description, color references are made to The Royal Horticultural Society Colour Chart, 2007 Edition, except where general terms of ordinary dictionary significance are used.
Botanical classification: Argyranthemum frutescens ‘SUN 480’.
Parentage:
   Female, or seed, parent.—Unnamed seedling selection of Argyranthemum frutescens, not patented.
   Male, or pollen, parent.—Unnamed seedling selection of Argyranthemum frutescens, not patented.
Propagation:
   Type.—Terminal cuttings.
   Time to initiate roots, summer.—About 8 to 10 days at temperatures about 18° C. to 24° C.
   Time to initiate roots, winter.—About 10 to 15 days at temperatures about 14° C. to 18° C.
   Time to produce a rooted young plant, summer.—About 14 to 16 days at temperatures about 18° C. to 24° C.
   Time to produce a rooted young plant, winter.—About 16 to 20 days at temperatures about 14° C. to 18° C.
   Root description.—Fine to medium in thickness, fibrous; typically white in color, actual color of the roots is dependent on substrate composition, water quality, fertilizer type and formulation, substrate temperature and physiological age of roots.
   Rooting habit.—Moderate branching; medium density.
Plant description:
   Plant and growth habit.—Herbaceous perennial; uniformly mounding plant habit; upright to broadly spreading form; overall shape, roughly flattened globular; moderately vigorous growth habit.
   Branching habit.—Freely branching growth habit with about ten lateral branches developing per plant; pinching enhances lateral branch development.
   Plant height.—About 14.1 cm.
   Plant width.—About 25.6 cm.
   Lateral branches.—Length: About 8.1 cm. Diameter: About 4 mm. Internode length: About 9 mm. Strength: Strong; young stems, flexible. Texture: Smooth, glabrous. Luster: Slightly glossy. Color: Close to between 144C and 145A; axial stripes, close to 144A.
Leaf description:
   Arrangement.—Alternate, simple; sessile.
   Length.—About 6.4 cm.
   Width.—About 4 cm.
   Shape.—Pinnatisect; in outline, obovate.
   Apex.—Acute.
   Base.—Cuneate.
   Margin.—Deeply incised.
   Sinuses.—Convergent.
   Texture, upper and lower surfaces.—Smooth, glabrous; leathery.
   Luster, upper and lower surfaces.—Matte.
   Venation.—Pinnate. Color: Developing leaves, upper surface: Close to 143A. Developing leaves, lower surface: Close to 143C. Fully expanded leaves, upper surface: Close to N137C; venation, close to 137C. Fully expanded leaves, lower surface: Close to 137C; venation, close to 137C.
Inflorescence description:
   Inflorescence form and arrangement.—Single-type terminal and axillary inflorescences held above and beyond the foliar plane on strong peduncles; ray and disc florets arranged acropetally on a receptacle; inflorescences face mostly upright to slightly outwardly.
   Flowering habit.—Freely flowering habit with about 140 inflorescences developing per plant during the flowering season.
   Flowering season.—Plants flower from spring to late summer in Germany; flowering continuous during this period.
   Inflorescence longevity.—Inflorescences last about two to three weeks on the plant; inflorescences not persistent.
   Fragrance.—Moderately fragrant.
   Inflorescence buds.—Height: About 7 mm. Diameter: About 8 mm. Shape: Flattened globular. Color: Towards the base, close to 143C; towards the apex, close to 199D; towards the margins, close to 165C.
   Inflorescence size.—Diameter: About 4.3 cm. Depth (height): About 1.4 cm. Diameter of disc: About 1.5 cm. Receptacle diameter: About 5 mm. Receptacle height: About 3 mm.
   Ray florets.—Quantity per inflorescence and arrangement: About 21 arranged in about two whorls. Length: About 1.7 cm. Width: About 6 mm. Shape: Oblong. Apex: Emarginate to shallowly praemorse. Base: Broadly attenuate. Margin: Entire. Texture, upper surface: Smooth, glabrous; velvety. Texture, lower surface: Glabrous; slightly carinate. Luster, upper surface: Matte. Luster, lower surface: Slightly glossy. Color: When opening, upper surface: Close to 61A to 61C tinged with close to N155B and 70A. When opening, lower surface: Close to 68B. Fully opened, upper surface: Close to between 53A and 59A, closer to 53A; with development, color becoming closer to between 53A and 59A, closer to 59A. Fully opened, lower surface: Close to 60C; color does not change with development.
   Disc florets.—Quantity per inflorescence and arrangement: About 200 spirally arranged at center of receptacle. Length: About 5 mm. Diameter, apex: About 2 mm. Diameter, base: About 0.5 mm. Shape: Tubular with five free apical lobes. Apex: Acute. Texture, inner and outer surfaces: Smooth, glabrous. Luster, inner surface: Glossy. Luster, outer surface: Matte. Color, immature, inner and outer surfaces: Apex: Close to 178A. Mid-section: Close to 150C. Base: Close to 150D. Color, mature, inner and outer surfaces: Apex: Close to 176A. Mid-section: Close to 145C to 145D. Base: Close to 145D.
   Phyllaries.—Quantity per inflorescence and arrangement: About 24 arranged in two whorls. Length: About 4 mm. Width: About 2.5 mm. Shape: Ovate. Apex: Praemorse. Base: Broadly cuneate. Margin: Entire. Texture, upper and lower surfaces: Smooth, glabrous; edges, papery. Luster, upper surface: Glossy. Luster, lower surface: Matte. Color, upper surface: Close to 144C to 144D; margins and papery edges, close to 199D. Color, lower surface: Close to 143B to 143C; margins and papery edges, close to 199D.
   Peduncles.—Length, terminal peduncle: About 3.4 cm. Length, fourth peduncle: About 6.3 cm. Diameter: About 2 mm. Angle: Terminal peduncle, erect; lateral peduncles, about 40° from branch axis. Strength: Strong. Texture: Slightly ribbed, glabrous. Luster: Slightly glossy. Color: Close to 138B.
   Reproductive organs.—Androecium: Present on disc florets only. Filament length: About 1.5 mm. Anther length: About 1 mm. Anther shape: Lanceolate; basifixed. Anther color: Close to 12A. Pollen amount: Scarce to moderate. Pollen color: Close to 14A. Gynoecium: Present on both ray and disc florets. Pistil length: About 3.5 mm. Stigma shape: Decurrent. Stigma color: Close to 12B. Style length: About 3 mm. Style color: Close to 145B. Ovary color: Close to 145C.
   Seeds and fruits.—Seed and fruit production has not been observed on plants of the new Argyranthemum.
Disease & pest resistance: Plants of the new Argyranthemum have not been shown to be resistant to pathogens and pests common to Argyranthemum plants.
Garden performance: Plants of the new Argyranthemum have been observed to have good garden performance and to tolerate rain, wind, high temperatures about 40° C. and to be hardy to USDA Hardiness Zone 9.

It is claimed:

1. A new and distinct Argyranthemum plant named ‘SUN 480’ as illustrated and described.
